(19) United States
(12) Patent Application Publication
(10) Pub. No.: US 2026/0079098 A1
(43) Pub. Date: Mar. 19, 2026

(54) ADHESIVE FORCE CONFIRMATION DEVICE

(71) Applicant: OMRON HEALTHCARE Co., Ltd., Kyoto (JP)

(72) Inventors: Makoto FUJIMOTO, Kyoto (JP); Tomoyuki NISHIDA, Kyoto (JP); Yoshiyuki TSUJI, Kyoto (JP)

(21) Appl. No.: 19/395,707

(22) Filed: Nov. 20, 2025

Related U.S. Application Data
(63) Continuation of application No. PCT/JP2024/002351, filed on Jan. 26, 2024.

(30) Foreign Application Priority Data
Sep. 22, 2023 (JP) 2023-158141

(51) Int. Cl. G01N 19/04; A61N 1/04

(52) U.S. Cl. CPC G01N 19/04; A61N 1/0472

(57) ABSTRACT

An adhesive force confirmation device is a device that confirms adhesive force of an electrode and is to be used for an electrical treatment device and includes an attaching portion to which an adhesive portion included in the electrode is attachable. The attaching portion includes a reference surface and working surfaces connected to the reference surface. The action surfaces are configured such that in a state where the adhesive portion is attached to the action surfaces, the adhesive portion is easily peeled off from sides of the action surfaces farther from the reference surface than from sides of the action surfaces closer to the reference surface.

### CROSS-REFERENCE TO RELATED APPLICATIONS

This application is the U.S. national stage application filed pursuant to 35 U.S.C. 365(c) and 120 as a continuation of International Patent Application No. PCT/JP2024/002351, filed Jan. 26, 2024, which application claims priority to Japanese Patent Application No. 2023-158141, filed Sep. 22, 2023, which applications are incorporated herein by reference in their entireties.

### FIELD

The present disclosure relates to an adhesive force confirmation device that confirms adhesive force of an electrode and is to be used for an electrical treatment device.

### BACKGROUND ART

Examples of a known method for evaluating peeling of an adhesive polymer gel, JP 2012-72262 A (Patent Document 1) include a method of attaching the gel to a glass plate, leaving it to stand for 24 hours at 60° C. under an atmosphere of 90% RH, and visually observing the presence or absence of foaming of the gel and the presence or absence of peeling at an end portion of the attached surface.

### SUMMARY OF INVENTION

### Technical Problem

An electrical treatment device such as a low-frequency treatment device alleviates muscle tension and pain by attaching an adhesive electrode to an affected area of a user and applying low-frequency electrical stimulation. The electrode is a consumable item whose adhesive force to the human body deteriorates due to contamination, adhesion of sweat, and the like caused by repeated use. The user does not know the expiration date of the electrode or continues to use the electrode for a longer period of time than the original specifications, whereby the original performance of the electrode may not sufficiently be exhibited.

A cordless low-frequency treatment device and an electrode that can be used while on the go have recently been developed, use in an environment where the electrode is likely to deteriorate is inevitable, and thus the need for the user to recognize the deterioration state of the electrode has been increasing.

However, using the evaluation method described in Patent Document 1 requires a thermostatic bath for maintaining a high-temperature and high-humidity environment for a predetermined period of time, and it is difficult for a user to easily confirm the deterioration state of the electrode (specifically, the adhesive force of the electrode) in everyday life.

The present disclosure has been made in view of the problem described above, and an object of the present disclosure is to provide an adhesive force confirmation device that can confirm adhesive force of an electrode with a simple configuration.

### Solution to Problem

An adhesive force confirmation device according to the present disclosure is a device that is for confirming adhesive force of an electrode and is to be used for an electrical treatment device. The adhesive force confirmation device includes an attaching portion to which an adhesive portion included in the electrode is attachable. The attaching portion includes a reference surface and a working surface connected to the reference surface. The working surface is configured such that in a state where the adhesive portion is attached to working surface, the adhesive portion is easily peeled off from a side of the working surface farther from the reference surface than from a side of the working surface closer to the reference surface.

In the adhesive force confirmation device according to the present disclosure, a direction perpendicular to the reference surface is defined as a height direction, and an end portion of the working surface located on the side farther from the reference surface may be located at a position lower than an end portion of the working surface located on the side closer to the reference surface.

In the adhesive force confirmation device according to the present disclosure, the working surface may include a curved portion that curves toward a lower position with increasing distance from the reference surface.

In the adhesive force confirmation device according to the present disclosure, the curved portion may be disposed having a decreasing radius of curvature with increasing distance from the reference surface.

In the adhesive force confirmation device according to the present disclosure, the working surface may include a stepped portion toward a lower position from the side closer to the reference surface toward the side farther from the reference surface.

The adhesive force confirmation device according to the present disclosure may further include a confirmation unit capable of confirming an amount of peeling of the electrode.

In adhesive force confirmation device according to the present disclosure, the confirmation unit may include a transparent portion through which at least a portion of the electrode peeled off from the attaching portion is visible and a scale indicating an amount of peeling from the attaching portion.

In the adhesive force confirmation device according to the present disclosure, the confirmation unit may include a sensor capable of detecting the amount of peeling.

In the adhesive force confirmation device according to the present disclosure, a part of the electrical treatment device may be provided in a fixable manner.

Advantageous Effects of Invention The present disclosure can provide an adhesive force confirmation device that can confirm adhesive force of an electrode with a simple configuration.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a perspective view illustrating a state of arranging an electrical treatment device on an adhesive force confirmation device according to a first embodiment.

FIG. 2 is a perspective view illustrating a state where the electrical treatment device is arranged on the adhesive force confirmation device according to the first embodiment.

FIG. 3 is a front view illustrating a state where the electrical treatment device is arranged on the adhesive force confirmation device according to the first embodiment.

FIG. 4 is a front view illustrating a state where electrodes are attached to working surfaces of the adhesive force confirmation device according to the first embodiment.

FIG. 5 is a perspective view illustrating a state where the electrical treatment device is arranged on an adhesive force confirmation device according to a second embodiment.

FIG. 6 is a diagram illustrating a confirmation unit that confirms the amount of peeling of the electrode in the adhesive force confirmation device according to the second embodiment.

FIG. 7 is a front view illustrating a state where the electrical treatment device is arranged on an adhesive force confirmation device according to a third embodiment.

FIG. 8 is a front view illustrating a state where the electrical treatment device is arranged on an adhesive force confirmation device according to a fourth embodiment.

FIG. 9 is a perspective view illustrating an adhesive force confirmation device according to a fifth embodiment.

FIG. 10 is a perspective view illustrating an adhesive force confirmation device according to a sixth embodiment.

FIG. 11 is a front view illustrating a state where the electrical treatment device is arranged on an adhesive force confirmation device according to a seventh embodiment.

FIG. 12 is a perspective view illustrating a state where an electrical treatment device is arranged on the adhesive force confirmation device according to an eighth embodiment.

### DESCRIPTION OF EMBODIMENTS

Embodiments of the present disclosure will be described in detail below with reference to the drawings. In the embodiments described below, identical or common components are denoted by the same reference numerals in the drawings, and repeated descriptions thereof are omitted.

### First Embodiment

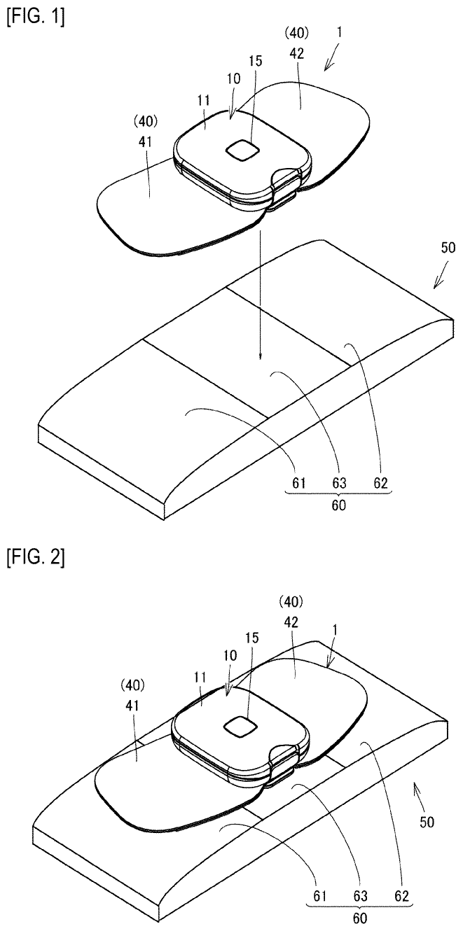

FIG. 1 is a perspective view illustrating a state of arranging an electrical treatment device on an adhesive force confirmation device according to a first embodiment. FIG. 2 is a perspective view illustrating a state where the electrical treatment device is arranged on the adhesive force confirmation device according to the first embodiment.

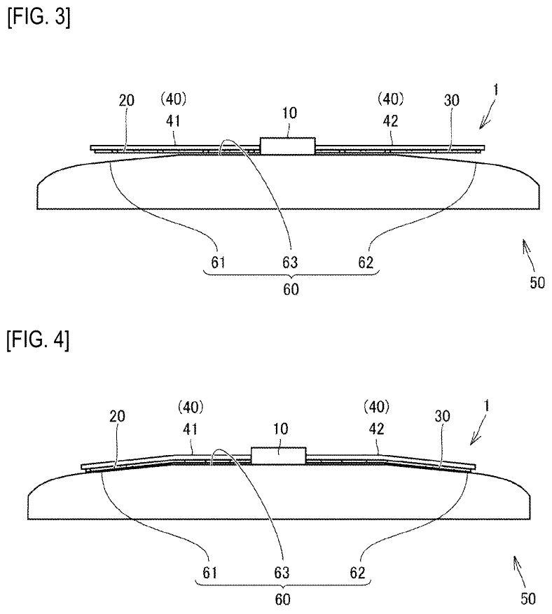

An adhesive force confirmation device 50 according to the first embodiment is a device that confirms adhesive force of two electrodes 20 and 30 (see FIG. 3) and is to be used for an electrical treatment device 1.

The electrical treatment device 1 includes a main body portion 10, electrodes 20 and 30 (see FIG. 3), and a holding portion 40. The electrical treatment device 1 is, for example, a low-frequency treatment device that relieves pain of a user by supplying a low-frequency pulse current to the electrode 20 and the electrode 30 that are in contact with a body surface of the user. The frequency of low-frequency pulse current is, for example, 1 Hz or higher and 1200 Hz or lower.

The main body portion 10 includes a housing case 11 and an operation unit 15. A circuit unit (not illustrated) is housed inside the housing case 11. The circuit unit is electrically connected to the electrodes 20 and 30 via an electrical path, and controls a current, a voltage, and the like flowing through the electrodes 20 and 30.

The operation unit 15 is disposed on the housing case 11. The operation unit 15 includes, for example, a measurement switch and the like. The operation unit 15 is not limited to a push type switch and may be, for example, a pressure sensitive type (resistance type) or a proximity type (capacitance type) touch panel type switch. The housing case 11 may be provided with a display unit separately from the operation unit 15.

The electrodes 20 and 30 are spaced apart from each other in a first direction and disposed such that the main body portion 10 is located between the electrode 20 and the electrode 30 in a plan view. The electrode 20 is disposed on one side in the first direction with respect to the main body portion 10. The electrode 30 is disposed on the other side in the first direction with respect to the main body portion 10.

The electrodes 20 and 30 are portions for applying a current to the user. The electrodes 20 and 30 are disposed to be attachable with adhesive force to the body surface of the user. The electrodes 20 and 30 include adhesive portions. The electrodes 20 and 30 may be, for example, gel pads or may be laminate bodies each constituted by an adhesive layer and a conductive member. By supplying a current to the electrodes 20 and 30 in a state where the electrodes 20 and 30 are in contact with the body surface of the user, the current can be applied to the user.

The holding portion 40 holds the electrodes 20 and 30. The holding portion 40 has, for example, flexibility and is disposed to be deformable along the body surface. For example, a cloth or the like having insulating properties can be employed as the holding portion 40.

The holding portion 40 and the electrodes 20 and 30 have flexibility and a restoring force acting to restore the original shapes when deformed, and as described below, peeling force can thus effectively act on the electrodes 20 and 30 (more specifically, the adhesive portions above) when the electrodes 20 and 30 are attached to the adhesive force confirmation device 50.

The holding portion 40 is fixed to, for example, the main body portion 10. The holding portion 40 includes a first holding portion 41 and a second holding portion 42. The first holding portion 41 holds the electrode 20. The shape of the first holding portion 41 is substantially identical to the shape of the electrode 20. The second holding portion 42 holds the electrode 30. The shape of the second holding portion 42 is substantially identical to the shape of the electrode 30. The first holding portion 41 and the second holding portion 42 may be connected by a connection portion.

The adhesive force confirmation device 50 includes an attaching portion 60 to which the two electrodes 20 and 30 (more specifically, the adhesive portions above) are attachable. In the first embodiment, the adhesive force confirmation device 50 has a block shape, and the attaching portion 60 is formed by an upper surface portion of the block shape. The adhesive force confirmation device 50 includes, for example, a resin member. The material, the surface roughness, the surface microstructure, and the like of the adhesive force confirmation device 50 are not particularly limited as long as the adhesive portions of the electrodes 20 and 30 are attachable to the device.

The attaching portion 60 includes two working surfaces 61 and 62 and a reference surface 63. The reference surface 63 is disposed to be, for example, flat. The reference surface 63 may be parallel to, for example, a horizontal direction, but the orientation may be changed as appropriate when measuring adhesive force.

The working surfaces 61 and 62 are connected to the reference surface 63. The working surfaces 61 and 62 are arranged such that the reference surface 63 is located between the working surfaces 61 and 62 in a plan view. The working surface 61, the reference surface 63, and the working surface 62 are arranged in this order in the first direction. The working surface 61 is disposed on one side in the first direction with respect to the reference surface 63. The working surface 62 is disposed on the other side in the first direction with respect to the reference surface 63.

The working surfaces 61 and 62 are configured such that in an attached state where the electrodes 20 and 30 are respectively attached to the working surfaces 61 and 62, the adhesive portions of the electrodes 20 and 30 are respectively and easily peeled off from a side of the working surfaces 61 and 62 farther from the reference surface 63 than from a side of the working surfaces 61 and 62 closer to the reference surface 63.

Specifically, when a direction perpendicular to the reference surface 63 is defined as a height direction, an end portion of each of the working surfaces 61 and 62, which is located on the side farther from the reference surface 63 is disposed at a position lower than an end portion of each of the working surfaces 61 and 62, which is located on the side closer to the reference surface 63. Note that when the reference surface 63 is parallel to the horizontal direction, the height direction is parallel to an up-down direction.

More specifically, when the direction perpendicular to reference surface 63 is defined as the height direction, the working surfaces 61 and 62 each have a curved portion that curves toward the lower position with increasing distance from the reference surface 63. The curved portion is disposed having a decreasing radius of curvature with increasing distance away from the reference surface 63.

The working surfaces 61 and 62 and the reference surface 63 are formed of a composition approximate to that of the body surface. For example, the working surfaces 61 and 62 and the reference surface 63 have surface roughness substantially similar to that of the body surface. Accordingly, the adhesiveness of the electrodes 20 and 30 to the body surface can be appropriately evaluated. The working surfaces 61 and 62 and the reference surface 63 may be formed of artificial skin.

At the time of arranging the electrical treatment device 1 on the adhesive force confirmation device 50, the electrical treatment device 1 is brought close to the adhesive force confirmation device 50 from above in a state where the main body portion 10 faces the reference surface 63 and the electrodes 20 and 30 face the working surfaces 61 and 62, and the main body portion 10 is placed on the reference surface 63.

FIG. 3 is a front view illustrating a state where the electrical treatment device is arranged on the adhesive force confirmation device according to the first embodiment.

As illustrated in FIG. 3, in a state where the main body portion 10 is placed on the reference surface 63, the electrodes 20 and 30 are separated from the attaching portion 60. In this state, the electrodes 20 and 30 are attached to the working surfaces 61 and 62.

FIG. 4 is a front view illustrating a state where the electrodes are attached to the working surfaces of the adhesive force confirmation device according to the first embodiment.

The electrode 20 described above includes a distal end portion located on the side farther from the main body portion 10 in the first direction and a base portion located on the side closer to the main body portion 10, and the distal end portion side of the electrode 20 is attached to the working surface 61. The base portion side of the electrode 20 may be attached to the reference surface 63.

The electrode 30 described above includes a distal end portion located on the side farther from the main body portion 10 in the first direction and a base portion located on the side closer to the main body portion 10, and the distal end portion side of the electrode 30 is attached to the working surface 62. The base portion side of the electrode 30 may be attached to the reference surface 63.

In the attached state described above, peeling force acts on the distal end portions of the electrodes 20 and 30 so as to return to the state illustrated in FIG. 3. In other words, the peeling force acts on the distal end portions of the electrodes 20 and 30 so that the holding portion 40 separates from the working surfaces 61 and 62. When the adhesive force of the electrodes 20 and 30 (more specifically, the adhesive portions) are weakened due to long-term use or the like, the electrodes 20 and 30 separate from the working surfaces 61 and 62 according to the adhesive force. By visually checking the degree of separation, the user can confirm the adhesive force of the electrodes 20 and 30.

The peeling force described above acts, for example, when an electrode pad including the electrodes 20 and 30 and the holding portion 40 is restored by elastic force. The elastic force of the electrode pad is weak or the initial adhesive force of the adhesive portions is strong, and the amount of peeling of the electrodes 20 and 30 is small even when the adhesive force is weakened due to long-term use. In such a case, stress may be applied in a direction opposite to the adhesive force. Specifically, the reference surface 63 may be directed downward or slightly sideways to cause gravity to act on the electrodes 20 and 30. Elastic members that generate stress against the adhesive force may be provided on the working surfaces 61 and 62. The elastic members are provided on the working surfaces 61 and 62 so as to face the respective distal end sides of the electrodes 20 and 30 in the attached state. When the elastic members are provided on the working surfaces 61 and 62, the working surfaces 61 and 62 and the reference surface 63 may be flat to each other and may be located on the same plane. Even when the electrodes 20 and 30 and the holding portion 40 do not have flexibility, the aforementioned elastic members may be provided.

As described above, in the adhesive force confirmation device 50 according to the present embodiment, by attaching the electrodes 20 and 30 to the working surfaces 61 and 62 and visually confirming an uplifted state of the electrodes 20 and 30, the adhesive force of the electrodes 20 and 30 can be confirmed. As a result, the adhesive force of the electrodes 20 and 30 can be confirmed by a simple configuration.

Note that, in the above description, a case where the electrodes 20 and 30 are attached to the working surfaces 61 and 62 after the main body portion 10 is placed on the reference surface 63 is illustrated, but the electrodes 20 and 30 may be attached to the working surfaces 61 and 62 at the same time as the main body portion 10 is placed on the reference surface 63.

### Second Embodiment

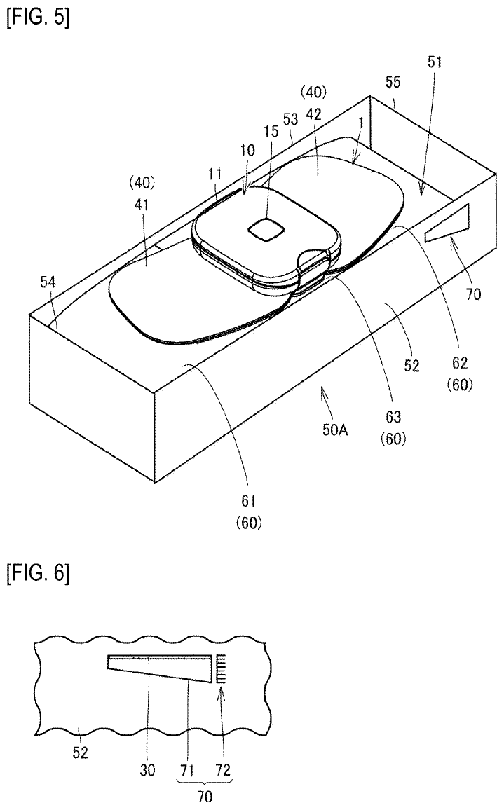

FIG. 5 is a perspective view illustrating a state where the electrical treatment device is arranged on an adhesive force confirmation device according to a second embodiment. An adhesive force confirmation device 50A according to the second embodiment will be described with reference to FIG. 5.

As illustrated in FIG. 5, the adhesive force confirmation device 50A according to the second embodiment is different from the adhesive force confirmation device 50 according to the first embodiment mainly in that a confirmation unit 70 capable of confirming the amount of peeling of the electrode is provided. Other configurations are almost the same.

The adhesive force confirmation device 50A includes the main body portion 51 and a plurality of wall portions 52 to 55. The main body portion 51 has a block shape and has substantially the same shape as that of the adhesive force confirmation device 50 of the first embodiment. The plurality of wall portions 52 to 55 are disposed so as to surround the main body portion 51. The plurality of wall portions 52 to 55 are disposed so as to protrude above the attaching portion 60.

The wall portions 54 and 55 are disposed so as to face each other in the first direction. The wall portion 54 is disposed at an end portion of the main body portion 51 located on one side in the first direction. The wall portion 55 is disposed at an end portion of the main body portion 51 located on the other side in the first direction.

The wall portions 52 and 53 are disposed so as to face each other in a second direction orthogonal to the first direction. The wall portion 52 is disposed at an end portion of the main body portion 51 located on one side in the second direction. The wall portion 53 is disposed at an end portion of the main body portion 51 located on the other side in the second direction. The second direction is orthogonal also to the up-down direction. Note that the up-down direction is parallel to a direction in which the adhesive force confirmation device 50A and the electrical treatment device 1 are located side by side in a state where the electrical treatment device 1 is arranged on the adhesive force confirmation device 50A. In addition, the up-down direction is a direction perpendicular to the reference surface 63 when the reference surface 63 is parallel to the horizontal direction. The confirmation unit 70 is disposed on the wall portion 52.

FIG. 6 is a diagram illustrating the confirmation unit that confirms the amount of peeling of the electrode in the adhesive force confirmation device according to the second embodiment.

The confirmation unit 70 includes a transparent portion 71 and a scale 72. The transparent portion 71 is disposed to allow the distal end portion of the electrode 30 to be visually recognized. For example, an acrylic plate can be adopted as the transparent portion 71. The scale 72 is disposed beside the transparent portion 71, and indicates the amount of separation of the distal end portion from the working surface 62. Note that the scale 72 may be disposed on the transparent portion 71.

In general, as the number of uses increases or the duration of use elapses, the adhesive force of the electrode 30 weakens with time. The position of the scale indicated by the distal end portion of the electrode 30 changes according to the intensity of the adhesive force. When the strong adhesive force is maintained, the distal end portion of the electrode 30 is located at the lowest position. As the adhesive force weakens, the distal end portion of the electrode 30 points to a scale located further on the upper side. As just described, the amount of peeling of the electrode 30 can be confirmed with the scale 72. Note that in the above description, a case where the direction of the scale is a direction perpendicular to the reference surface 63 is illustrated, but the direction is not limited thereto, and may be a direction parallel to the reference surface 63.

With such a configuration as above, the adhesive force confirmation device 50A according to the second embodiment can achieve effects similar to those in the first embodiment. The confirmation unit 70 is provided, and thus the current adhesive force can be more accurately confirmed.

### Third Embodiment

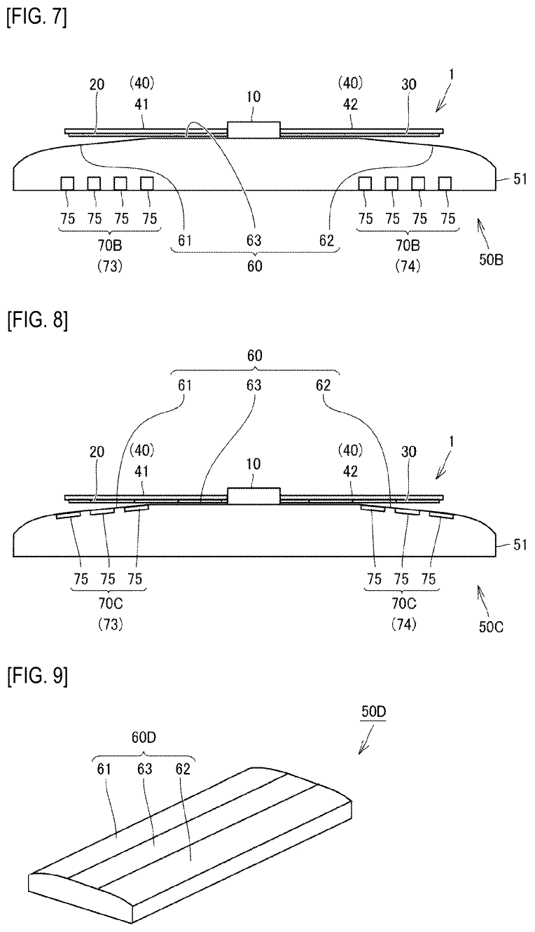

FIG. 7 is a front view illustrating a state where the electrical treatment device is arranged on an adhesive force confirmation device according to a third embodiment. An adhesive force confirmation device 50B according to the third embodiment will be described with reference to FIG. 7.

As illustrated in FIG. 7, the adhesive force confirmation device 50B according to the third embodiment is different from the adhesive force confirmation device 50 according to the first embodiment mainly in that a confirmation unit 70B capable of confirming the amount of peeling of the electrodes is provided. Other configurations are almost the same.

The confirmation unit 70B includes a first unit 73 that measures the amount of peeling of the electrode 20 and a second unit 74 that measures the amount of peeling of the electrode 30. The first unit 73 and the second unit 74 each include a plurality of distance sensors 75. The plurality of distance sensors 75 are, for example, arranged side by side in the first direction. The plurality of distance sensors 75 are arranged so as to be located below the electrodes 20 and 30 in a state where the electrodes 20 and 30 are attached. The plurality of distance sensors 75 are, for example, embedded in the main body portion 51.

Each of the plurality of distance sensors 75 is provided so as to be able to measure a distance from the distance sensor 75 to the electrode 30 along the up-down direction. For example, ultrasonic sensors capable of detecting the distance to an object in a non-contact manner can be adopted as the plurality of distance sensors 75.

As the adhesive force weakens, the distance from the distance sensor 75 to the electrode 30 increases. By measuring the distance, the amount of peeling of the electrodes 30 can be confirmed.

With such a configuration as above, the adhesive force confirmation device 50B according to the third embodiment can achieve effects similar to those in the first embodiment. The confirmation unit 70B is provided, and thus the current adhesive force can be more accurately confirmed.

### Fourth Embodiment

FIG. 8 is a front view illustrating a state where the electrical treatment device is arranged on an adhesive force confirmation device according to a fourth embodiment. An adhesive force confirmation device 50C according to the fourth embodiment will be described with reference to FIG. 8.

As illustrated in FIG. 8, the adhesive force confirmation device 50C according to the fourth embodiment is different from the adhesive force confirmation device 50 according to the first embodiment mainly in that a confirmation unit 70C capable of confirming the amount of peeling of the electrodes is provided. Other configurations are almost the same.

The confirmation unit 70C includes the first unit 73 that measures the amount of peeling of the electrode 20 and the second unit 74 that measures the amount of peeling of the electrode 30. The first unit 73 and the second unit 74 each include a plurality of distance sensors 75. The plurality of distance sensors 75 are, for example, arranged side by side in the first direction. The plurality of distance sensors 75 are arranged so as to be located below the electrodes 20 and 30 in a state where the electrodes 20 and 30 are attached. The plurality of distance sensors 75 may be disposed, for example, on the working surfaces 61 and 62.

Each of the plurality of distance sensors 75 is, for example, a contact type electrode sensor, and can measure the impedance (resistance value), the electric capacitance, or the like of the electrodes 20 and 30. The amount of peeling of the electrodes 20 and 30 can be confirmed by measuring the impedance or the like that changes according to the contact state of the electrodes 20 and 30.

With such a configuration as above, the adhesive force confirmation device 50C according to the fourth embodiment can achieve effects similar to those in the first embodiment. The confirmation unit 70C is provided, and thus the current adhesive force can be more accurately confirmed.

### Fifth Embodiment

FIG. 9 is a perspective view illustrating an adhesive force confirmation device according to a fifth embodiment. An adhesive force confirmation device 50D according to the fifth embodiment will be described with reference to FIG. 9.

As illustrated in FIG. 9, the adhesive force confirmation device 50D according to the fifth embodiment is different from the adhesive force confirmation device 50 according to the first embodiment in that the shape of an attaching portion 60D is different. Other configurations are almost the same.

In the attaching portion 60D, the working surface 61, the reference surface 63, and the working surface 62 are arranged side by side in this order in the second direction. The working surface 61 is disposed on one side in the second direction with respect to the reference surface 63. The working surface 62 is disposed on the other side in the second direction with respect to the reference surface 63.

In this case, an end portion of the electrodes 20 and 30, which is located on the one side in the second direction is attached to the working surface 61, and an end portion of the electrodes 20 and 30, which is located on the other side in the second direction is attached to the working surface 62.

With such a configuration as above, the adhesive force confirmation device 50D according to the fifth embodiment can achieve effects similar to those in the first embodiment.

### Sixth Embodiment

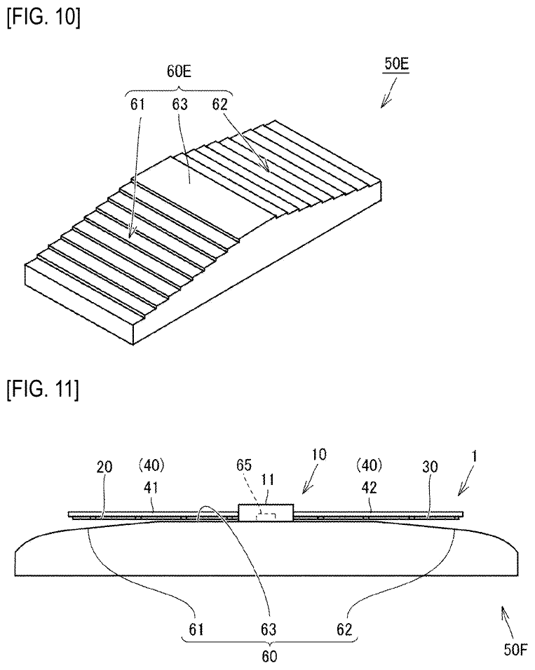

FIG. 10 is a perspective view illustrating an adhesive force confirmation device according to a sixth embodiment. An adhesive force confirmation device 50E according to the sixth embodiment will be described with reference to FIG. 10.

As illustrated in FIG. 10, the shape of the adhesive force confirmation device 50E according to the sixth embodiment is different from the adhesive force confirmation device 50 according to the first embodiment in that the shape of an attaching portion 60E is different. Other configurations are almost the same.

In the attaching portion 60E, each of the working surfaces 61 and 62 includes a stepped portion disposed toward a lower position from the side closer to the reference surface 63 toward the side farther from the reference surface 63.

With such a configuration as above, the adhesive force confirmation device 50E according to the sixth embodiment can achieve effects similar to those in the first embodiment.

### Seventh Embodiment

FIG. 11 is a front view illustrating a state the electrical treatment device is arranged on an adhesive force confirmation device according to a seventh embodiment. An adhesive force confirmation device 50F according to the seventh embodiment will be described with reference to FIG. 11.

As illustrated in FIG. 11, the adhesive force confirmation device 50F according to the seventh embodiment is different from the adhesive force confirmation device 50 according to the first embodiment in that a part of the electrical treatment device 1 is disposed so as in a fixable manner. Other configurations are almost the same.

The adhesive force confirmation device 50F according to the seventh embodiment is provided with an engagement portion 65. The engagement portion 65 is disposed, for example, on the reference surface 63. The engagement portion 65 is disposed so as to be engageable with the main body portion 10. The engagement portion 65 is, for example, a protrusion protruding from the reference surface 63. The protrusion is disposed so as to be fitted into an engagement recessed portion disposed in the main body portion 10. The engagement portion 65 is engaged with the main body portion 10, thereby positioning the main body portion 10. Thus, the electrical treatment device 1 can be prevented from moving while the adhesive force of the electrodes 20 and 30 is being confirmed.

### Eighth Embodiment

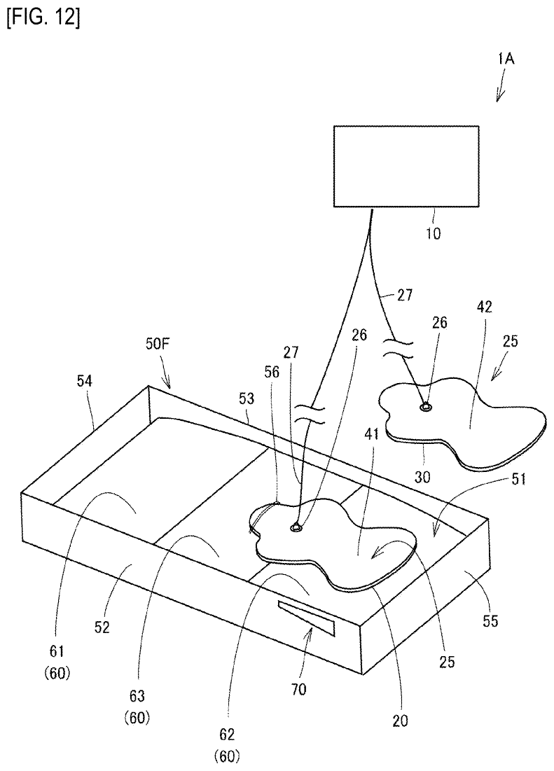

FIG. 12 is a perspective view illustrating a state where an electrical treatment device is arranged on the adhesive force confirmation device according to an eighth embodiment. The adhesive force confirmation device 50F according to the eighth embodiment will be described with reference to FIG. 12.

As illustrated in FIG. 12, the adhesive force confirmation device 50F according to the eighth embodiment is different from the adhesive force confirmation device 50D according to the fifth embodiment in that a fixing tool 56 is provided. In addition, an electrical treatment device 1A including the main body portion 10, two electrode pads 25, and two electrode cords 27 may be used as the electrical treatment device.

The main body portion 10 is located separate from the two electrode pads 25, and the two electrode pads 25 are respectively connected to the main body portion 10 by the electrode cords 27. The electrode cords 27 each include a connector portion 26 and can be detachably attached to the electrode pad 25. One electrode pad 25 includes the aforementioned electrode 20 and first holding portion 41, and the other electrode pad 25 includes the aforementioned electrode 30 and second holding portion 42. The first holding portion 41 and the second holding portion 42 are formed independently of each other, and the two electrode pads 25 are disposed so as to be movable independently of each other.

In such a case, as illustrated in FIG. 12, only one of the electrode pads 25 may be attached to the adhesive force confirmation device 50F. In this case, the electrode pad 25 is attached so as to extend from the reference surface 63 to the working surface 61 or the working surface 62. In this case, a part of the electrical treatment device 1A (specifically, a part of the electrode pad 25) may be fixed to the fixing tool 56. The fixing tool 56 is disposed on the reference surface 63. For example, a strap-shaped member such as a rubber band or a cord may be used as the fixing tool 56. In this case, an end portion of the electrode pad 25 is pressed against the reference surface 63 by the strap-shaped member. Note that in place of the strap-shaped member, an adhesive tape or the like may be used as the fixing tool 56. Alternatively, the fixing tool 56 may be omitted, and the user may press the end portion of the electrode pad 25 against the reference surface 63 to fix the electrode pad 25.

Although a case where the adhesive force confirmation device 50F is attached in a state where the electrode pad 25 is connected to the electrode cord 27 is illustrated in FIG. 12, only the electrode pad 25 may be attached in a state where the electrode cord 27 is removed.

### Other Modified Examples

The adhesive force confirmation device may include a first surface and a second surface that face each other, and may be provided with the electrode 20 attached to the first surface and the electrode 30 attached to the second surface in a state where the holding portion 40 is bent in a substantially U-shape.

In the above description, a case where the peeling force is adjusted by providing the difference in height between the working surfaces 61 and 62 is illustrated, but not limited thereto, and the peeling force may be adjusted by performing surface treatment on the working surfaces 61 and 62. For example, the surface roughness of the working surfaces 61 and 62 on the side farther from the reference surface 63 may be smaller than the surface roughness of the working surfaces 61 and 62 on the side closer to the reference surface 63.

### SUPPLEMENTARY NOTES

As described above, the present embodiment includes the following disclosures.
Configuration 1: An adhesive force confirmation device that confirms adhesive force of an electrode and is to be used for an electrical treatment device, the adhesive force confirmation device including an attaching portion to which an adhesive portion included in the electrode is attachable, wherein the attaching portion includes a reference surface and a working surface connected to the reference surface, and the working surface is configured such that in a state where the adhesive portion is attached to the working surface, the adhesive portion is easily peeled off from a side of the working surface farther from the reference surface than from a side of the working surface closer to the reference surface.
Configuration 2: The adhesive force confirmation device according to Configuration 1, wherein a direction perpendicular to the reference surface is defined as a height direction, and an end portion of the working surface located on the side farther from the reference surface is located at a position lower than an end portion of the working surface located on the side closer to the reference surface.
Configuration 3: The adhesive force confirmation device according to Configuration 2, wherein the working surface includes a curved portion that curves toward a lower position with increasing distance from the reference surface.
Configuration 4: The adhesive force confirmation device according to Configuration 3, wherein the curved portion is disposed having a decreasing radius of curvature with increasing distance from the reference surface.
Configuration 5: The adhesive force confirmation device according to Configuration 2, wherein the working surface includes a stepped portion toward a lower position from the side closer to the reference surface toward the side farther from the reference surface.
Configuration 6: The adhesive force confirmation device according to any one of Configurations 1 to 5, further including a confirmation unit capable of confirming an amount of peeling of the electrode.
Configuration 7: The adhesive force confirmation device according to Configuration 6, wherein the confirmation unit includes a transparent portion through which at least a portion of the electrode peeled off from the attaching portion is visible and a scale indicating an amount of peeling from the attaching portion.
Configuration 8: The adhesive force confirmation device according to Configuration 6, wherein the confirmation unit includes a sensor capable of detecting the amount of peeling.
Configuration 9: The adhesive force confirmation device according to any one of Configurations 1 to 8, wherein a part of the electrical treatment device is provided in a fixable manner.

The embodiments disclosed herein are illustrative in all respects and are not intended as limitations. The scope of the present invention is defined by the claims, and all modified examples within the meaning and scope equivalent to the claims are intended to be included therein.

### REFERENCE SIGNS LIST

1 Electrical treatment device, 10 Main body portion, 11 Housing case, 15 Operation unit, 20 and 30 Electrode, 25 Electrode pad, 26 Connector portion, 27 Electrode cord, 40 Holding portion, 41 First holding portion, 42 Second holding portion, 50, 50A, 50B, 50C, 50D, 50E, 50F Adhesive force confirmation device, 51 Main body portion, 52, 53, 54, 55 Wall portion, 56 Fixing tool, 60, 60D, 60E Attaching portion, 61, 62 Working surface, 63 Reference surface, 65 Engagement portion, 70, 70B, 70C Confirmation unit, 71 Transparent portion, 72 Scale, 73 First unit, 74 Second unit, 75 Distance sensor

1. An adhesive force confirmation device that confirms adhesive force of an electrode and is to be used for an electrical treatment device, the adhesive force confirmation device comprising
an attaching portion to which an adhesive portion included in the electrode is attachable, wherein
the attaching portion comprises a reference surface and a working surface connected to the reference surface, and
the working surface is configured such that in a state where the adhesive portion is attached to the working surface, the adhesive portion is easily peeled off from a side of the working surface farther from the reference surface than from a side of the working surface closer to the reference surface.

2. The adhesive force confirmation device according to claim 1, wherein
a direction perpendicular to the reference surface is defined as a height direction, and
an end portion of the working surface located on the side farther from the reference surface is located at a position lower than an end portion of the working surface located on the side closer to the reference surface.

3. The adhesive force confirmation device according to claim 2, wherein the working surface comprises a curved portion that curves toward a lower position with increasing distance from the reference surface.

4. The adhesive force confirmation device according to claim 3, wherein the curved portion is disposed having a decreasing radius of curvature with increasing distance from the reference surface.

5. The adhesive force confirmation device according to claim 2, wherein the working surface comprises a stepped portion toward a lower position from the side closer to the reference surface toward the side farther from the reference surface.

6. The adhesive force confirmation device according to claim 1, further comprising
a confirmation unit capable of confirming an amount of peeling of the electrode.

7. The adhesive force confirmation device according to claim 6, wherein the confirmation unit comprises a transparent portion through which at least a portion of the electrode peeled off from the attaching portion is visible and a scale indicating an amount of peeling from the attaching portion.

8. The adhesive force confirmation device according to claim 6, wherein the confirmation unit comprises a sensor capable of detecting the amount of peeling.

9. The adhesive force confirmation device according to claim 1, wherein a part of the electrical treatment device is provided in a fixable manner.
